(19) United States
(12) Patent Application Publication
(10) Pub. No.: US 2019/0269306 A1
(43) Pub. Date: Sep. 5, 2019

(54) FIXING DEVICE FOR ENDOSCOPE

(71) Applicants: KYUSHU UNIVERSITY, NATIONAL UNIVERSITY CORPORATION, Fukuoka-shi, Fukuoka (JP); HOGY MEDICAL CO., LTD., Tokyo (JP)

(72) Inventors: Ryu NAKADATE, Fukuoka-shi (JP); Makoto HASHIZUME, Fukuoka-shi (JP); Shunsuke NAGAI, Tokyo (JP); Hiroyasu FUJITA, Tokyo (JP); Jiro KATO, Tokyo (JP)

(73) Assignees: KYUSHU UNIVERSITY, NATIONAL UNIVERSITY CORPORATION, Fukuoka-shi, Fukuoka (JP); HOGY MEDICAL CO., LTD., Tokyo (JP)

(21) Appl. No.: 16/344,969

(22) PCT Filed: Aug. 25, 2017
(86) PCT No.: PCT/JP2017/030530

§ 371 Date: Apr. 25, 2019

(30) Foreign Application Priority Data
Oct. 26, 2016 (JP) 2016-210030

(51) Int. Cl. A61B 1/00

(52) U.S. Cl. CPC A61B 1/00147

(57) ABSTRACT

A fixing device for an endoscope that allows easy detachment of the endoscope from an endoscope holder even with the endoscope kept inserted in a body cavity is provided.

A fixing device for fixing an endoscope capable of observing an operative site in a body cavity using an endoscope having an insertion portion to be inserted into the body cavity and an endoscope body attached at an end of the insertion portion. The fixing device includes a holder body which is attached to a fixing stand placed on a floor and holds the endoscope body in a rotatable manner. The holder body includes a detaching mechanism that allows the endoscope body to be detached from the holder body while keeping the insertion portion inserted in the body cavity.

### TECHNICAL FIELD

The present invention relates to a fixing device for an endoscope that holds a medical appliance used in a surgery, such as an endoscope, in place of a surgeon.

### BACKGROUND ART

A laparoscopic surgery with an endoscope is typically performed by two persons: an endoscope operator who operates an endoscope for observing the interior of the abdominal cavity and a surgeon who conducts the surgery while viewing an image of the abdominal cavity captured by the endoscope. During the surgery, the endoscope operator continues holding the endoscope and also performs operations such as changing the orientation of the endoscope or advancing and retreating it in the insertion direction via oral instructions or the like when the surgeon wants to change the observation location at the operative site or the angle of view during the surgery. For this type of surgery method, communication between the surgeon and the endoscope operator is very important. Due to issues associated with space in an operation room and/or the necessity for smooth communication as just mentioned, there is a demand for the ability to hold and fix of an endoscope so that the surgeon can also operate the endoscope.

Various methods are known for such fixing of an endoscope. For example, an endoscope holder having an arm for holding an endoscope, a supporting portion on which an endoscope holder is fixed, and multiple joints is known, such as one described in Patent Literature 1. The arm, the supporting portion and the joints are respectively equipped with handles, and the arm, the supporting portion and the joints are fixed and released by operating the handles so that the endoscope is held in a movable or fixable manner.

More recently, approaches like endoscopic submucosal dissection (ESD) have been also put into practice. The ESD involves inserting a treatment instrument through the mouth, the anus and the like and removing a piece of surface layer of mucosa over a wide area of the stomach or the large intestine without piercing through the wall of the stomach or the large intestine. Further, an approach called Natural Orifice Translumenal Endoscopic Surgery (NOTES) is known. The NOTES involves inserting a flexible endoscope such as a stomach or intestine camera through the mouth, the anus, the vagina or the urethra, which are naturally present in the body surface, further delivering the flexible endoscope to the abdominal cavity piercing through the wall of the stomach or the large intestine, and performing diagnosis or treatment of an organ in the abdominal cavity.

Such translumenal endoscopic surgery represented by the endoscopic submucosal dissection (ESD) performs treatment and the like by inserting treatment instruments such as forceps and a knife along with a flexible endoscope through the mouth or some other opening naturally present in the body surface, and delivering them to the site of disease. Thus, it can reduce invasion to the human body by causing no damage to the body surface and eliminating complication such as infection or adhesion of the abdominal wall, which can occur in a traditional surgery.

A treatment instrument for use in such a translumenal endoscopic surgery has a bending portion for bendably operating the treatment instrument as inserted in a flexible endoscope and projecting from a tip of the flexible endoscope, as described in Patent Literature 2. The treatment instrument also has a sheath wire portion for transmitting bending motions to the bending portion, and an operation input portion for operating the bending motion of the bending portion by pushing and pulling of the sheath wire.

### CITATION LIST

### Patent Literature

### Patent Literature 1: Japanese Patent Laid-Open No. 2003-325436

### Patent Literature 2: Japanese Patent Laid-Open No. 2010-511440

### SUMMARY OF INVENTION

### Technical Problem

However, the endoscope holder described in Patent Literature 1 does not allow the endoscope to be removed from the endoscope holder while the endoscope is inserted in a body cavity because it fixes the endoscope by inserting it into a through hole formed in the arm. This leads to the problem of detachment of the endoscope being very difficult when it is necessary to remove the endoscope from the endoscope holder for some reason and operate it by the endoscope operator as in conventional practice.

This problem, or the difficulty in detaching the endoscope, is further complicated in the case of ESD because ESD is performed with a bending treatment instrument inserted in the endoscope and thus an increased number of items are carried on the endoscope.

The present invention was made to solve such a problem; specifically, an object thereof is to provide a fixing device for an endoscope that allows easy detachment of the endoscope from an endoscope holder even with the endoscope kept inserted in a body cavity.

### Solution to Problem

A fixing device according to the present invention for attaining the object is a fixing device for fixing an endoscope capable of observing an operative site in a body cavity using an endoscope having an insertion portion to be inserted into the body cavity and an endoscope body attached at an end of the insertion portion. The fixing device includes a holder body attached to a fixing stand placed on a floor and for holding the endoscope body in an axially rotatable manner, and the holder body includes a detaching mechanism that allows the endoscope body to be detached from the holder body while keeping the insertion portion inserted in the body cavity.

In the fixing device according to the present invention, the detaching mechanism preferably includes: a through hole formed in the holder body and having a first cutout in a radial direction; and a turning component rotatably fitted in the through hole and having a second cutout corresponding to the first cutout.

The fixing device according to the present invention preferably includes a pressing device which presses the turning component in an axial direction of the through hole and has a third cutout corresponding to the first cutout and the second cutout.

In the fixing device according to the present invention, the turning component preferably includes a flange extending in the radial direction, and the pressing device preferably presses the flange in the axial direction.

In the fixing device according to the present invention, the turning component and the pressing device are preferably fitted in the through hole and fixed in the axial direction by a lid component attached on the holder body.

In the fixing device according to the present invention, the holder body preferably includes a horn component for fixing a treatment instrument which is inserted into an endoscope channel of the endoscope or inserted into the body cavity with the endoscope.

### Advantageous Effect of Invention

With the present invention, the endoscope body is fixed to the fixing device having a detaching mechanism, which allows the endoscope body to be detached from the fixing device while keeping the endoscope inserted in a body cavity. Thus, it is possible to remove the endoscope from the fixing device and immediately switch to an approach in which the endoscope is operated by the endoscope operator as in the conventional practice when it is necessary to do so for some reason.

### BRIEF DESCRIPTION OF DRAWINGS

FIG. 1 is a schematic illustration for describing how a fixing device for an endoscope according to an embodiment is used.

FIG. 2 is a schematic illustration for generally describing a surgery method with bending treatment instruments.

FIG. 3 is a side view for describing the structure of a bending treatment instrument for use in an endoscopic surgery.

FIG. 4 is a perspective view of a fixing stand to which the fixing device according to the embodiment is attached.

FIG. 5 shows the fixing device according to the embodiment with an endoscope attached.

FIG. 6 is a perspective view of the fixing device according to the embodiment.

FIG. 7 is a perspective view of a holder body of the fixing device according to the embodiment.

FIG. 8 is an exploded view of the holder body of the fixing device according to the embodiment.

### DESCRIPTION OF EMBODIMENT

A fixing device for an endoscope according to the present invention is described below with reference to the drawings. The embodiment described below is not intended to limit the subject matters set forth in the claims and not all of the combinations of features described in the embodiment are essential for the solution of the invention.

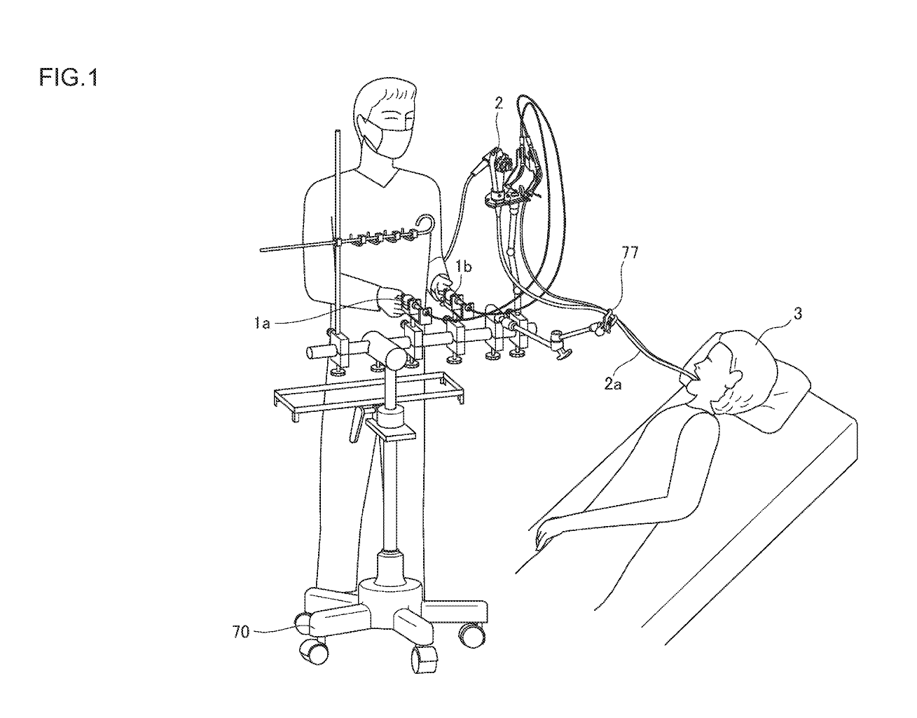

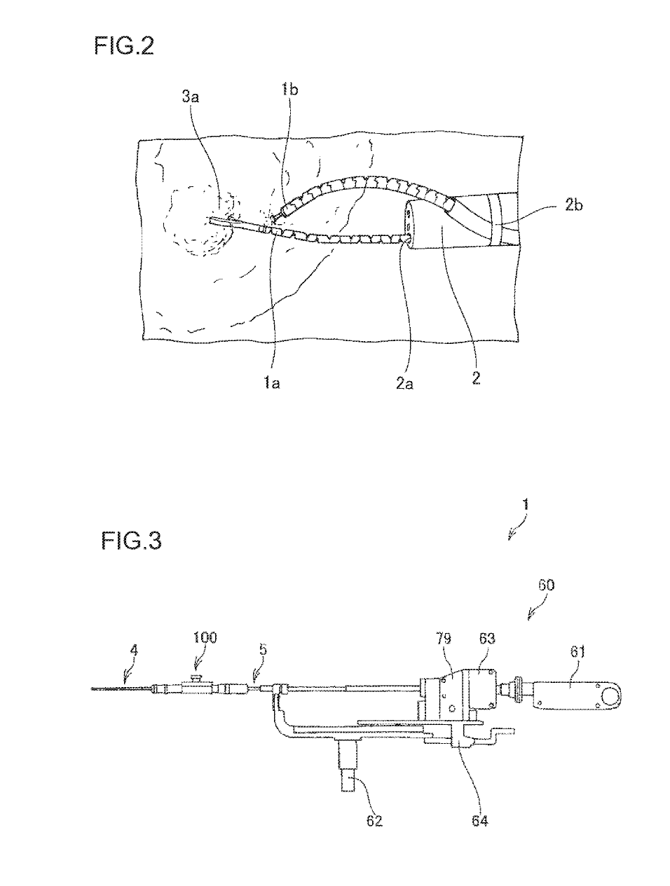

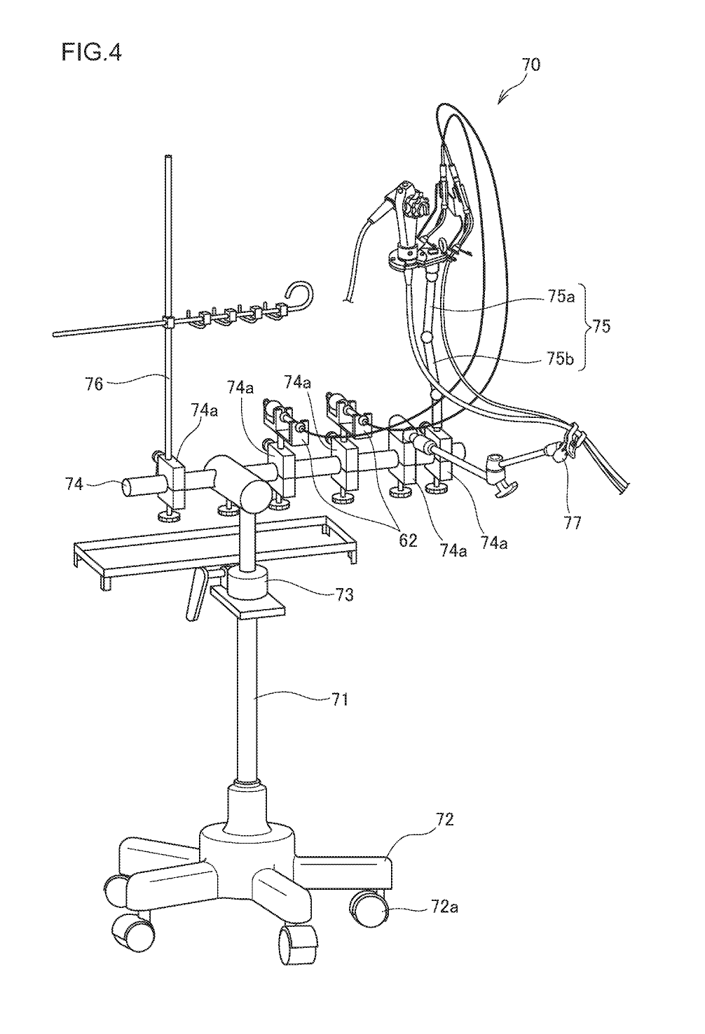

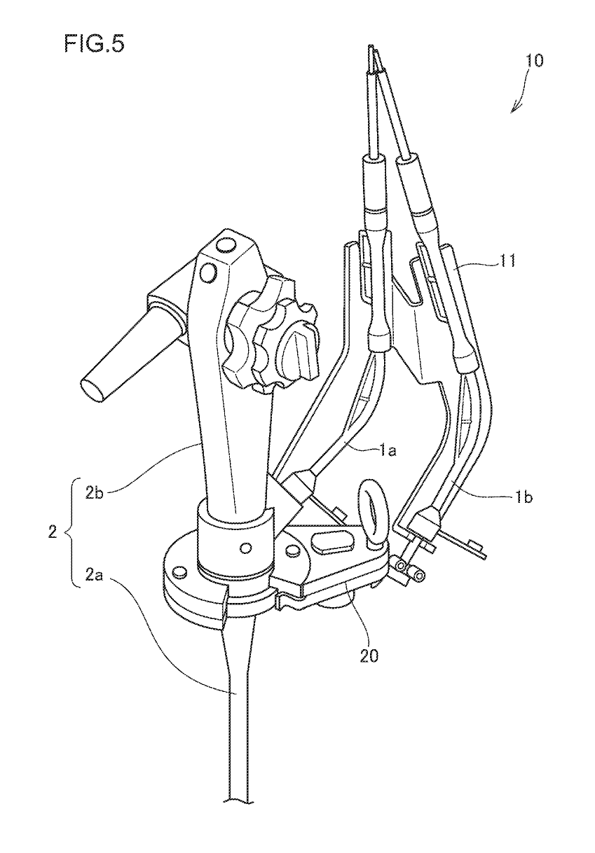

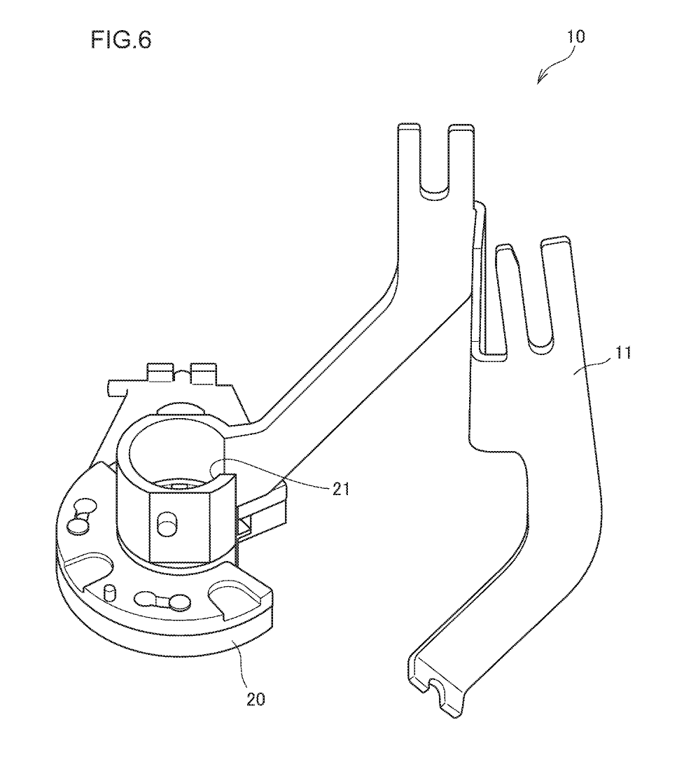

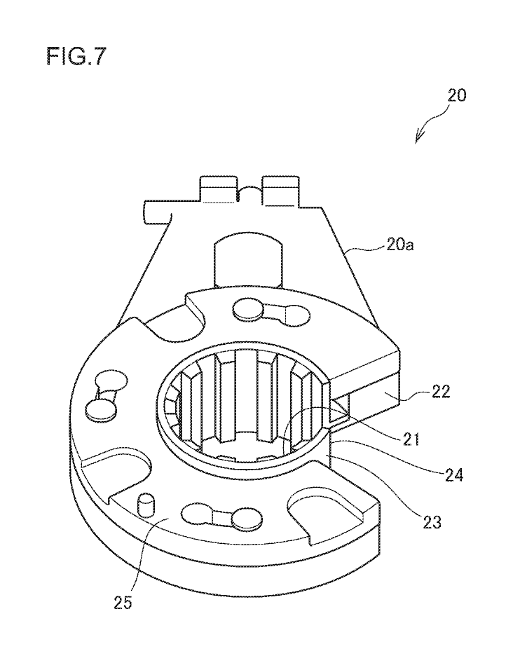

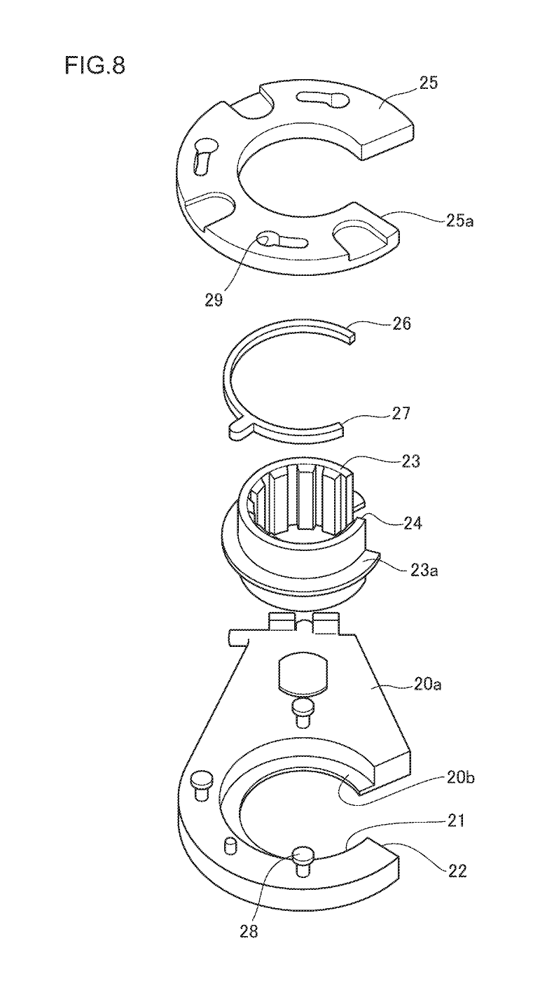

FIG. 1 is a schematic illustration for describing how a fixing device for an endoscope according to an embodiment is used; FIG. 2 is a schematic illustration for generally describing a surgery method with bending treatment instruments; FIG. 3 is a side view for describing the structure of a bending treatment instrument for use in an endoscopic surgery; FIG. 4 is a perspective view of a fixing stand to which the fixing device according to the embodiment is attached; FIG. 5 shows the fixing device according to the embodiment with an endoscope attached; FIG. 6 is a perspective view of the fixing device according to the embodiment; FIG. 7 is a perspective view of a holder body of the fixing device according to the embodiment; and FIG. 8 is an exploded view of the holder body of the fixing device according to the embodiment.

As shown in FIGS. 1 and 2, in a laparoscopic surgery with an endoscope, an insertion portion 2a of a flexible endoscope 2 is inserted into a patient 3 through his/her mouth, anus or the like. Then, diagnosis is made on an affected area 3a, such as a cancer in an abdominal cavity like a digestive organ, using images of the interior of the abdominal cavity captured by the flexible endoscope 2. Alternatively, the affected area 3a is removed using a bending treatment instrument 1a inserted into an endoscope channel and a bending treatment instrument 1b which is attached to the insertion portion 2a by a treatment instrument insertion tube 2b attached to the insertion portion 2a and is inserted into the patient from his/her mouth, anus or the like along with the insertion portion 2a. For the bending treatment instruments 1a and 1b, a bending treatment instrument for forceps 1a equipped with forceps at its tip and a bending treatment instrument for knife 1b equipped with an electric knife at its tip are advantageously used.

The bending treatment instrument for forceps 1a and the bending treatment instrument for knife 1b bend individually and independently from the flexible endoscope 2 so as to have at least two-degree-of-freedom. It is thus possible to grip or exsect the affected area 3a while fixing the point of view of the flexible endoscope 2, enabling a procedure with a stable field of view and high degree of freedom. As noted above, the bending treatment instrument for forceps 1a and the bending treatment instrument for knife 1b are different in the component attached to their tips. Thus, they will be referred to as bending treatment instrument 1 in the following description; the bending treatment instrument for forceps 1a and the bending treatment instrument for knife 1b are to be encompassed by the bending treatment instrument 1.

As shown in FIG. 3, the bending treatment instrument 1 includes forceps which are attached to the tip of a bending portion 4 having two-degree-of-freedom in the horizontal and vertical directions and are fitted in an openable and closeable manner by a device wire as discussed below, or an electric knife (not shown). The bending treatment instrument 1 also includes an operation input portion 60 for making bending motions of the bending portion 4, opening and closing motions of the forceps, and advancing and retracting of the electric knife, and a sheath wire portion 5 including multiple wires for transmitting the operation of the operation input portion 60 and a sheath into which the wires are inserted.

The operation input portion 60 is attached on a fixing stand connector 62 via a direct acting device 64, which is capable of sliding an operation input portion body 63 in the longitudinal direction. The operation input portion 60 allows the forceps, the bending portion 4, and the sheath wire portion 5 to be pushed and pulled along the longitudinal direction by sliding the direct acting device 64 in the longitudinal direction, thereby adjusting the amount of projection of the forceps from the endoscope channel 2c or from the treatment instrument insertion tube 2b. The fixing stand connector 62 is attached on a fixing stand 70. As the bending treatment instrument 1 is a well-known treatment instrument, it is not described in detail herein.

As shown in FIG. 4, a fixing device 10 according to this embodiment is used by being attached to the fixing stand 70. The fixing stand 70 includes a fixing stand body 71, multiple legs 72 attached to one end of the fixing stand body 71, and a fixing base 74 attached at the other end of the fixing stand body 71 via a height adjustment mechanism 73. Each of the legs 72 has a caster 72a attached thereon, being configured to facilitate the movement of the fixing stand 70. Preferably, the casters 72a are configured to permit locking and unlocking of rolling. Such a configuration can prevent an unintended movement of the fixing stand 70 while the fixing stand 70 is in use.

The fixing base 74 is a rod-shaped component extending from the fixing stand body 71 substantially in the horizontal direction. A joint component 75, the fixing stand connector 62, a hanger component 76, and an endoscope gripping arm 77 are each attached on the fixing base 74 via a fixing portion 74a.

The joint component 75 includes a first joint component 75a and a second joint component 75b, which are configured to be bendable so that the position of an endoscope body 2b attached to the fixing device 10 can be fixed at a desired position. The joint component may include two or more joint components if they can be configured to be bendable relative to one another. In this embodiment, the fixing device 10 is attached to one end of the first joint component 75a and a fixing portion 74a is attached to one end of the second joint component 75b, with their other ends being coupled with each other in a bendable manner.

The hanger component 76 is a component on which treatment instruments and the like used in the surgery are temporarily kept. By keeping sterilized treatment instruments and the like on it, they can be prevented from making contact with unclean objects such as other treatment instruments and the like, so that the sterilized state of the treatment instruments being kept on the hanger component can be maintained. For holding treatment instruments and the like on the hanger component 76, various methods may be applied. For example, the hanger component 76 may be advantageously configured to grip treatment instruments in a clipping fashion.

The endoscope gripping arm 77 is a component for gripping the insertion portion 2a in order to prevent it from falling off due to the self-weight of the flexible endoscope 2 or the insertion portion 2a even in a case where the flexible endoscope 2 is fixed to the fixing stand 70 by gripping the insertion portion 2a when the insertion portion 2a of the flexible endoscope 2 is inserted into the patient 3 through his/her mouth, anus and the like, as shown in FIG. 1. Since the insertion portion 2a is thus gripped by the endoscope gripping arm 77, a surgery can be performed by a single surgeon without requiring an endoscope operator by fixing the flexible endoscope 2 to the fixing stand 70. In this embodiment, the endoscope gripping arm 77 is described for a case where it has a clip structure and the insertion portion 2a is gripped by the clip structure. However, the tip structure of the endoscope gripping arm 77 is not limited to a clip structure and may be any of various structures that can appropriately fix the insertion portion 2a.

As shown in FIG. 5, the fixing device 10 of this embodiment is configured such that the endoscope body 2b of the flexible endoscope 2 can be fixed to the holder body 20. Specifically, a through hole into which the endoscope body 2b can be inserted is formed in the holder body 20, and the endoscope body 2b is held by inserting the endoscope body 2b into the through hole.

The fixing device 10 also has a horn component 11 attached on the holder body 20. By fixing the shaft fixing portions of the bending treatment instruments 1a and 1b to the horn component 11, the movement ranges of the bending treatment instruments 1a and 1b are limited so as not to hinder persons or objects when the flexible endoscope 2 is turned in order to handle the flexible endoscope 2.

As shown in FIG. 6, the fixing device 10 of this embodiment is provided with a detaching mechanism, which allows the endoscope body 2b to be detached from the holder body 20 while keeping the endoscope body 2b attached in the through hole 21 of the holder body 20 and the insertion portion 2a inserted in a body cavity.

Referring to FIGS. 7 and 8, the structures of the detaching mechanism and the holder body 20 are described. As shown in FIG. 7, the holder body 20 includes a holder body base 20a with the through hole 21 having a first cutout 22 formed therein, a turning component 23 attached in the through hole 21, a pressing device 26 for pressing the turning component 23 in the axial direction of the through hole 21, and a lid component 25 for fixing the turning component 23 and the pressing device 26 in the axial direction relative to the holder body base 20a.

At the through hole 21 of the holder body base 20a, a retainer portion 20b to engage with a flange 23a of the turning component 23 is formed. The turning component 23 is a substantially C-shaped tubular component with a second cutout 24 corresponding to the first cutout 22, and has the flange 23a extending in the radial direction formed on its outer peripheral surface. Since the turning component 23 turns by sliding against the holder body base 20a, it is preferably made of synthetic resin with heat resistance and high rigidity in order to deter abrasion caused by sliding and/or prevent creation of dust.

Also, the turning component 23 is pressed by the pressing device 26 in the axial direction, and the pressing device 26 has a third cutout 27 formed therein corresponding to the first cutout and the second cutout 24. The pressing device 26 may be any component that can press the flange 23a of the turning component 23 in the axial direction. For example, a wave washer or the like is advantageously used as an adjustment spring.

Further, the turning component 23 and the pressing device 26 are fixed in the axial direction by the lid component 25 attached to the holder body base 20a. The lid component 25 has a fourth cutout 25a corresponding to the first cutout 22, the second cutout 24 and the third cutout 27, and also has an engagement groove 29 formed therein. The engagement groove 29 is configured to be mate with an engaging protrusion 28 which is formed on the holder body base 20a so as to correspond to the engagement groove 29, so that the lid component 25 can be fixed to the holder body base 20a. In this case, the engagement groove 29 has an insertion part formed to be slightly larger than the diameter of the head of the engaging protrusion 28 and a fixing part formed to be smaller than the diameter of the head of the engaging protrusion 28. By then inserting the engaging protrusion 28 into the insertion part of the engagement groove 29 and rotating the lid component 25 in the circumferential direction, the engaging protrusion 28 is moved into the fixing part of the engagement groove 29, thereby fixing the holder body base 20a and the lid component 25 together.

Being thus configured, the fixing device 10 allows the turning motion of the endoscope body 2b fitted in the turning component 23 because the turning component 23 is fitted in the through hole 21 of the holder body 20. Additionally, since the turning component 23 is pressed in the axial direction by the pressing device 26, reasonable frictional resistance is given when the turning component 23 is turned, preventing the turning component 23 from turning under load as low as the self-weight of the endoscope body 2b. This enables holding of the endoscope body 2b without having a specific locking mechanism for the turning component 23.

Also, due to the formation of the first to fourth cutouts 22, 24, 27, 25a in the holder body 20, the turning component 23, the pressing device 26 and the lid component 25, it is easy to detach the endoscope body 2b from the holder body 20 by aligning the positions of the first to fourth cutouts 22, 24, 27, 25a even while the insertion portion 2a of the flexible endoscope 2 inserted in a body cavity.

As described above, the fixing device 10 of this embodiment is capable of fixing the flexible endoscope 2 and/or the bending treatment instruments 1a, 1b to the fixing stand 70 together. Thus, it allows a surgeon to perform a surgery on his own using bending treatment instruments without requiring an endoscope operator to hold the flexible endoscope. It also makes it possible to immediately remove the endoscope body 2b from the fixing stand 70 where necessary, such as in an emergency, and switch to a traditional, hand-holding type surgery with the endoscope operator.

While a preferred embodiment of the present invention has been described, the technical scope of the present invention is not limited to the above-described embodiment. Various modifications or improvement may be made to the embodiment.

The fixing device 10 according to the above embodiment was described for a case where the holder body base 20a and the lid component 25 are fixed to each other via the engaging protrusion 28 and the engagement groove 29. However, for example, engagement between the holder body base 20a and the lid component 25 is not limited to by the engagement of the engaging protrusion 28 and the engagement groove 29 but any fixing device that can fix them together may be used. For example, a well-known fixing device such as a clip or a bolt and nut may be employed or they may be fixed together by adhesive or welding.

It will be apparent from the description in the claims that forms with such modifications or improvements can fall within the technical scope of the present invention.

### REFERENCE SIGNS LIST

1a bending treatment instrument for forceps
1b bending treatment instrument for knife
2 flexible endoscope
2a insertion portion
2b endoscope body
2c endoscope channel
3 patient
3a affected area
4 bending portion
5 sheath wire portion
10 fixing device
11 horn component
20 holder body
20a holder body base
21 through hole
22 first cutout
23 turning component
23a flange
24 second cutout
25 lid component
25a fourth cutout
26 pressing device
27 third cutout
28 engaging protrusion
29 engagement groove
70 fixing stand
71 fixing stand body
72 leg
73 height adjustment mechanism
74 fixing base
75 joint component
76 hanger component

1. A fixing device for fixing an endoscope capable of observing an operative site in a body cavity using an endoscope having an insertion portion to be inserted into the body cavity and an endoscope body attached at an end of the insertion portion, wherein
the fixing device comprises a holder body attached to a fixing stand placed on a floor and for holding the endoscope body in an axially rotatable manner, and
the holder body comprises a detaching mechanism that allows the endoscope body to be detached from the holder body while keeping the insertion portion inserted in the body cavity.

2. The fixing device according to claim 1, wherein the detaching mechanism comprises:
a through hole formed in the holder body and having a first cutout in a radial direction; and
a turning component rotatably fitted in the through hole and having a second cutout corresponding to the first cutout.

3. The fixing device according to claim 2, comprising a pressing device which presses the turning component in an axial direction of the through hole and has a third cutout corresponding to the first cutout and the second cutout.

4. The fixing device according to claim 2, wherein the turning component comprises a flange extending in the radial direction, and the pressing device presses the flange in the axial direction.

5. The fixing device according to claim 4, wherein the turning component and the pressing device are fitted in the through hole and fixed in the axial direction by a lid component attached on the holder body.

6. The fixing device according to claim 1, wherein the holder body comprises a horn component for fixing a treatment instrument which is inserted into an endoscope channel of the endoscope or inserted into the body cavity with the endoscope.
